(19) United States
(12) Patent Application Publication
(10) Pub. No.: US 2016/0136446 A1
(43) Pub. Date: May 19, 2016

(54) WEARABLE CARDIAC DEFIBRILLATOR SYSTEM DELIVERING PROMPTS TO PATIENT

(71) Applicant: West Affum Holdings Corp., Grand Cayman (KY)

(72) Inventors: John Carlton Daynes, Redmond, WA (US); Robert Reuben Buchanan, Bothell, WA (US); Douglas Keith Medema, Everett, WA (US); Isabelle Banville, Redmond, WA (US)

(21) Appl. No.: 14/548,254

(22) Filed: Nov. 19, 2014

(51) Int. Cl. A61N 1/39

(52) U.S. Cl. CPC A61N 1/3968; A61N 1/3993

(57) ABSTRACT

A wearable cardiac defibrillator (“WCD”) system includes a speaker for issuing audio prompts to the patient and bystanders. The speaker may be located in a head component such as a headset or glasses or an earpiece or a pendant, or be pressed against the body of the patient. The volume of the sound may be adjusted or muted.

### BACKGROUND

When people suffer from some types of heart arrhythmias, the result may be that blood flow to various parts of the body is reduced. Some arrhythmias may even result in a Sudden Cardiac Arrest (SCA). SCA can lead to death very quickly, e.g. within 10 minutes, unless treated in the interim.

Some people have an increased risk of SCA. People at a higher risk include individuals who have had a heart attack, or a prior SCA episode. A frequent recommendation is for these people to receive an Implantable Cardioverter Defibrillator (“ICD”). The ICD is surgically implanted in the chest, and continuously monitors the person's electrocardiogram (“ECG”). If certain types of heart arrhythmias are detected, then the ICD delivers an electric shock through the heart.

After being identified as having an increased risk of an SCA, and before receiving an ICD, these people are sometimes given a wearable cardiac defibrillator (“WCD”) system. A WCD system typically includes a harness, vest, or other garment for wearing by the patient. The system includes a defibrillator and external electrodes, which are attached on the inside of the harness, vest, or other garment. When a patient wears a WCD system, the external electrodes may then make good electrical contact with the patient's skin, and therefore can help monitor the patient's ECG. If a shockable heart arrhythmia is detected, then the defibrillator of the WCD system delivers the appropriate electric shock through the patient's body, and thus through the heart.

In a number of instances, a WCD system gives messages to the patient who is wearing it. The messages can include instructions, reports, reminders, and so on. The messages can be delivered verbally by a speaker, but sometimes there are problems in the delivery.

### BRIEF SUMMARY

The present description gives instances of wearable cardiac defibrillator (“WCD”) systems, software, and methods, the use of which may help overcome problems and limitations of the prior art. A WCD system may include a support structure that a patient can wear, an energy storage module that can store an electrical charge, and a discharge circuit that can discharge the electrical charge through the patient so as to shock him or her, while the patient is wearing the support structure. Embodiments may also include a speaker configured to output for the patient a sound that can include prompts, instructions, questions, and so on.

In embodiments, a WCD system may include a head component that includes the speaker, or a pendant that includes the speaker. In embodiments, a WCD system may have two speakers for outputting different prompts. In embodiments, a WCD system may have a microphone and a memory, and it starts recording ambient sounds upon recognizing that one or more preset words were said. In embodiments, the support structure of a WCD system may be such that it is worn by the patient under tension, and the tension physically biases the speaker towards the body of the patient.

In embodiments, a WCD system may include an interface that is configured to receive a volume adjustment input or a switching input from the patient. Accordingly, a volume of the sound may be adjusted, or a speaker can be muted. In some of the muting embodiments, upon the speaker being muted, another device can be enabled so as to provide the prompts to the patient in an alternative way.

These and other features and advantages of this description will become more readily apparent from the Detailed Description, which proceeds with reference to the associated drawings in which:

### BRIEF DESCRIPTION OF THE DRAWINGS

FIG. 1 is a diagram of components of a sample wearable cardiac defibrillator (WCD) system, made according to embodiments.

FIG. 2 is a diagram showing sample components of an external defibrillator, such as the one belonging in the WCD system of FIG. 1, and which is made according to embodiments.

FIG. 3 is a diagram showing a collection of sample components of Wearable Cardiac Defibrillator systems included in many embodiments.

FIG. 4 is a diagram of a patient wearing components of a sample wearable cardiac defibrillator (WCD) system that are made according to additional embodiments that include a head component.

FIG. 5 is a diagram of a patient wearing components of a sample wearable cardiac defibrillator (WCD) system that are made according to other embodiments that include a head component.

FIG. 6 is a diagram showing sample components of a personal communication device (PCD) that could be the PCD of FIG. 5, and which can be made according to embodiments.

FIG. 7 is a diagram of a patient wearing components of a sample wearable cardiac defibrillator (WCD) system that are made according to embodiments that include a neck pendant.

FIG. 8 is a diagram of selected components of a sample wearable cardiac defibrillator (WCD) system having at least two speakers that can be tasked differently according to embodiments.

FIG. 9 is a diagram of a patient wearing components of a sample wearable cardiac defibrillator (WCD) system that can be made according to embodiments, and in an example where a speaker is biased towards the body of the patient.

FIG. 10 is a diagram of a portion of sample user interface of a wearable cardiac defibrillator (WCD) system made according to an embodiment that permits adjusting a volume of a sound being output from a speaker.

FIG. 11 is a diagram of a portion of sample user interface of a wearable cardiac defibrillator (WCD) system made according to an embodiment that permits muting of a sound being output from a speaker.

FIG. 12 is a flowchart for illustrating methods according to embodiments.

### DETAILED DESCRIPTION

As has been mentioned, the present description is about wearable cardiac defibrillator (“WCD”) systems, software, and methods. Embodiments are now described in more detail.

A wearable cardiac defibrillator (WCD) system made according to embodiments has a number of components. These components can be provided separately as modules that can be interconnected, or can be combined with other components, etc.

A component of a WCD system can be a support structure, which is configured to be worn by the patient. The support structure can be any structure suitable for wearing, such as a harness, a vest, a half-vest—for example over the left side of the torso that positions electrodes on opposite sides of the heart, one or more belts that are configured to be worn horizontally or possibly vertically over a shoulder, another garment, and so on. The support structure can be implemented in a single component, or multiple components. For example, a support structure may have a top component resting on the shoulders, for ensuring that the defibrillation electrodes will be in the right place for defibrillating, and a bottom component resting on the hips, for carrying the bulk of the weight of the defibrillator. A single component embodiment could be with a belt around at least the torso. Other embodiments could use an adhesive structure or another way for attaching to the person, without encircling any part of the body. There can be other examples.

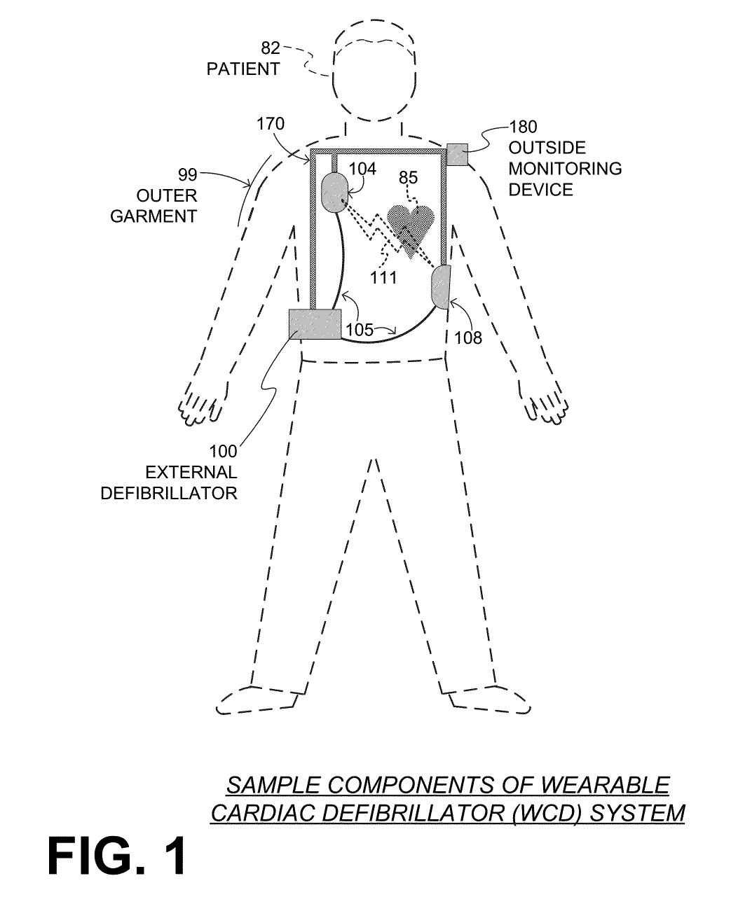

FIG. 1 depicts components of a WCD system made according to embodiments, as it might be worn by a person 82. A person such as person 82 may also be referred to as a patient and/or wearer, since that person wears components of the WCD system. Patient 82 may wear garments, one or more of which can be associated with a WCD system. In addition, a portion of an outer garment 99 is shown. Outer garment 99 might be obscuring the WCD system that patient 82 is wearing.

In FIG. 1, a generic support structure 170 is shown relative to the body of person 82, and thus also relative to his or her heart 85. Structure 170 could be a harness, a vest, a half-vest, one or more belts, or a garment, etc., as per the above. Structure 170 could be implemented in a single component, or multiple components, and so on. Structure 170 is wearable by person 82, but the manner of wearing it is not depicted, as structure 170 is depicted only generically in FIG. 1.

A wearable cardiac defibrillator (WCD) system is configured to defibrillate a patient who is wearing it, by delivering electrical charge to the patient's body in the form of an electric shock delivered in one or more pulses. FIG. 1 shows a sample external defibrillator 100, and sample defibrillation electrodes 104, 108, which are coupled to external defibrillator 100 via electrode leads 105. Defibrillator 100 and defibrillation electrodes 104, 108 are coupled to support structure 170. As such, many of the components of defibrillator 100 can be therefore coupled to support structure 170. When defibrillation electrodes 104, 108 make good electrical contact with the body of person 82, defibrillator 100 can administer, via electrodes 104, 108, a brief, strong electric pulse 111 through the body. Pulse 111, also known as a defibrillation shock or therapy shock, is intended to go through and restart heart 85, in an effort to save the life of person 82. Pulse 111 can further include one or more pacing pulses, and so on.

A prior art defibrillator typically decides whether to defibrillate or not based on an electrocardiogram (“ECG”) signal of the patient. However, defibrillator 100 can defibrillate, or not defibrillate, also based on other inputs.

The WCD system may optionally include an outside monitoring device 180. Device 180 is called an “outside” device because it is provided as a standalone device, for example not within the housing of defibrillator 100. Device 180 can be configured to monitor at least one local parameter. A local parameter can be a parameter of patient 82, or a parameter of the WCD system, or a parameter of the environment, as will be described later in this document.

Optionally, device 180 is physically coupled to support structure 170. In addition, device 180 can be communicatively coupled with other components, which are coupled to support structure 170. Such communication can be implemented by a communication module, as will be deemed applicable by a person skilled in the art in view of this disclosure.

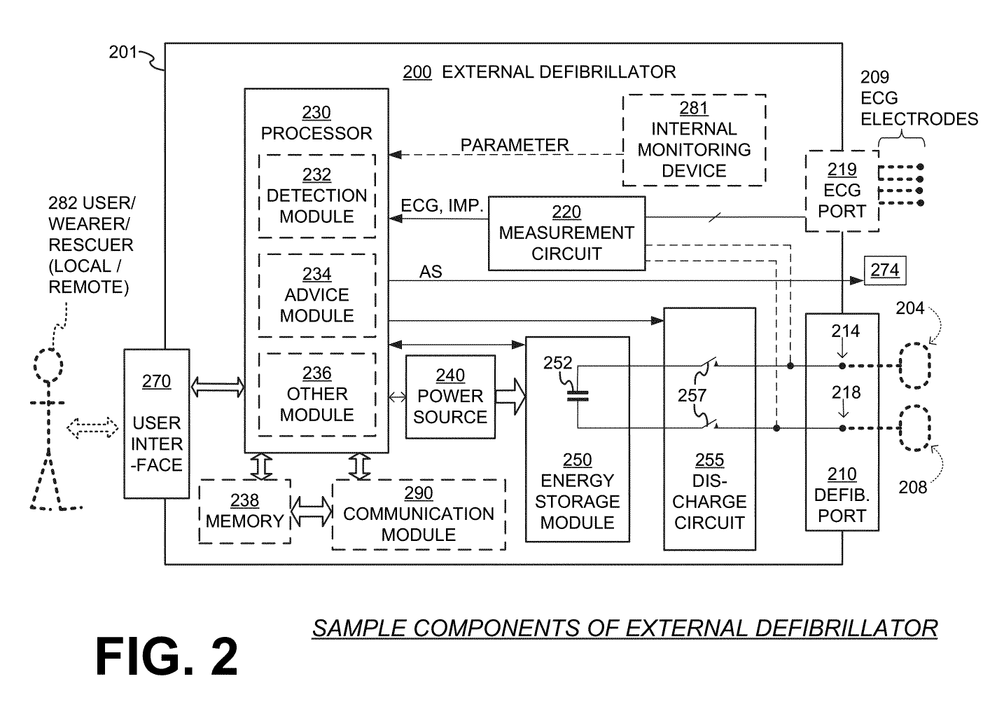

FIG. 2 is a diagram showing components of an external defibrillator 200, made according to embodiments. These components can be, for example, included in external defibrillator 100 of FIG. 1. The components shown in FIG. 2 can be provided in a housing 201, which is also known as casing 201.

External defibrillator 200 is intended for a patient who would be wearing it, such as person 82 of FIG. 1. Defibrillator 200 may further include a user interface 270 for a user 282. User 282 can be patient 82, also known as wearer 82. Or user 282 can be a local rescuer at the scene, such as a bystander who might offer assistance, or a trained person. Or, user 282 might be a remotely located trained caregiver in communication with the WCD system.

User interface 270 can be made in any number of ways. User interface 270 may include output devices, which can be visual, audible or tactile, for communicating to a user. For example, an output device can be a light, or a screen to display what is detected and measured, and provide visual feedback to rescuer 282 for their resuscitation attempts, and so on. Another output device can be a speaker, which can be configured to issue voice prompts, etc. Sounds, images, vibrations, and anything that can be perceived by user 282 can also be called human perceptible indications. User interface 270 may also include input devices for receiving inputs from users. Such input devices may include various controls, such as pushbuttons, keyboards, touchscreens, a microphone, validation input devices such as a keypad, a fingerprint reader, and so on. An input device can be a cancel switch, which is sometimes called a “live-man” switch. In some embodiments, actuating the cancel switch can prevent the impending delivery of a shock.

Defibrillator 200 may include an internal monitoring device 281. Device 281 is called an “internal” device because it is incorporated within housing 201. Monitoring device 281 can monitor patient parameters, patient physiological parameters, system parameters and/or environmental parameters, all of which can be called patient data. In other words, internal monitoring device 281 can be complementary or an alternative to outside monitoring device 180 of FIG. 1. Allocating which of the system parameters are to be monitored by which monitoring device can be done according to design considerations.

Patient physiological parameters include, for example, those physiological parameters that can be of any help in detecting by the WCD system whether the patient is in need of a shock, plus optionally their medical history and/or event history.

Defibrillator 200 typically includes a defibrillation port 210, such as a socket in housing 201. Defibrillation port 210 includes electrical nodes 214, 218. Leads of defibrillation electrodes 204, 208, such as leads 105 of FIG. 1, can be plugged in defibrillation port 210, so as to make electrical contact with nodes 214, 218, respectively. It is also possible that defibrillation electrodes 204, 208 are connected continuously to defibrillation port 210, instead. Either way, defibrillation port 210 can be used for guiding, via electrodes, to the wearer the electrical charge that has been stored in energy storage module 250. The electric charge will be the shock for defibrillation, pacing, and so on.

Defibrillator 200 may optionally also have an ECG port 219 in housing 201, for plugging in sensing electrodes 209, which are also known as ECG electrodes and ECG leads. It is also possible that sensing electrodes 209 can be connected continuously to ECG port 219, instead. Sensing electrodes 209 can help sense an ECG signal, e.g. a 12-lead signal, or a signal from a different number of leads, especially if they make good electrical contact with the body of the patient. Sensing electrodes 209 can be attached to the inside of support structure 170 for making good electrical contact with the patient, similarly as defibrillation electrodes 204, 208.

Defibrillator 200 also includes a measurement circuit 220. Measurement circuit 220 receives physiological signals of the patient from ECG port 219, if provided. Even if defibrillator 200 lacks ECG port 219, measurement circuit 220 can obtain physiological signals through nodes 214, 218 instead, when defibrillation electrodes 204, 208 are attached to the patient. In these cases, the patient's ECG signal can be sensed as a voltage difference between electrodes 204, 208. Plus, impedance between electrodes 204, 208 and/or the connections of ECG port 219 can be sensed. Sensing the impedance can be useful for detecting, among other things, whether these electrodes 204, 208 and/or sensing electrodes 209 are not making good electrical contact with the patient's body. These patient physiological signals can be sensed, when available. Measurement circuit 220 can then render or generate information about them as physiological inputs, data, other signals, etc. More strictly speaking, the information rendered by measurement circuit 220 is output from it, but this information can be called an input because it is received by a subsequent device or functionality as an input.

Defibrillator 200 also includes a processor 230. Processor 230 may be implemented in any number of ways. Such ways include, by way of example and not of limitation, digital and/or analog processors such as microprocessors and digital-signal processors (DSPs); controllers such as microcontrollers; software running in a machine; programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

Processor 230 can be considered to have a number of modules. One such module can be a detection module 232. Detection module 232 can include a ventricular fibrillation (“VF”) detector. The patient's sensed ECG from measurement circuit 220, which can be available as physiological inputs, data, or other signals, may be used by the VF detector to determine whether the patient is experiencing VF. Detecting VF is useful, because VF results in SCA. Detection module 232 can also include a ventricular tachycardia (“VT”) detector, and so on.

Another such module in processor 230 can be an advice module 234, which generates advice for what to do. The advice can be based on outputs of detection module 232. There can be many types of advice according to embodiments. In some embodiments, the advice is a shock/no shock determination that processor 230 can make, for example via advice module 234. The shock/no shock determination can be made by executing a stored Shock Advisory Algorithm. A Shock Advisory Algorithm can make a shock/no shock determination from one or more of ECG signals that are captured according to embodiments, and determining whether or not a shock criterion or shock condition is met. The determination can be made from a rhythm analysis of the captured ECG signal or otherwise.

In some embodiments, when the decision is to shock, an electrical charge is delivered to the patient. Delivering the electrical charge is also known as discharging. Shocking can be for defibrillation, pacing, and so on.

Processor 230 can include additional modules, such as other module 236, for other functions. In addition, if internal monitoring device 281 is indeed provided, it may be operated in part by processor 230, etc.

Defibrillator 200 optionally further includes a memory 238, which can work together with processor 230. Memory 238 may be implemented in any number of ways. Such ways include, by way of example and not of limitation, volatile memories, nonvolatile memories (NVM), read-only memories (ROM), random access memories (RAM), magnetic disk storage media, optical storage media, smart cards, flash memory devices, any combination of these, and so on. Memory 238 is thus a non-transitory storage medium. Memory 238, if provided, can include programs for processor 230, which processor 230 may be able to read, and execute. More particularly, the programs can include sets of instructions in the form of code, which processor 230 may be able to execute upon reading. Executing is performed by physical manipulations of physical quantities, and may result in the functions, processes, actions and/or methods to be performed, and/or the processor to cause other devices or components or blocks to perform such functions, processes, actions and/or methods. The programs can be operational for the inherent needs of processor 230, and can also include protocols and ways that decisions can be made by advice module 234. In addition, memory 238 can store prompts for user 282, if they are a local rescuer. Moreover, memory 238 can store data. The data can include patient data, system data and environmental data, for example as learned by internal monitoring device 281 and outside monitoring device 180. The data can be stored in memory 238 before it is transmitted out of defibrillator 200, or stored there after it is received by it.

Defibrillator 200 may also include a power source 240. To enable portability of defibrillator 200, power source 240 typically includes a battery. Such a battery is typically implemented as a battery pack, which can be rechargeable or not. Sometimes a combination is used of rechargeable and non-rechargeable battery packs. Other embodiments of power source 240 can include an AC power override, for where AC power will be available, an energy storage capacitor, and so on. In some embodiments, power source 240 is controlled by processor 230.

Defibrillator 200 additionally includes an energy storage module 250, which can thus be coupled to the support structure of the wearable system. Module 250 is where some electrical energy is stored in the form of an electrical charge, when preparing it for sudden discharge to administer a shock. Module 250 can be charged from power source 240 to the right amount of energy, as controlled by processor 230. In typical implementations, module 250 includes a capacitor 252, which can be a single capacitor or a system of capacitors, and so on. As described above, capacitor 252 can store the energy in the form of electrical charge, for delivering to the patient.

Defibrillator 200 moreover includes a discharge circuit 255. When the decision is to shock, processor 230 can be configured to control discharge circuit 255 to discharge through the patient the electrical charge stored in energy storage module 250. When so controlled, circuit 255 can permit the energy stored in module 250 to be discharged to nodes 214, 218, and from there also to defibrillation electrodes 204, 208. Circuit 255 can include one or more switches 257. Switches 257 can be made in a number of ways, such as by an H-bridge, and so on. Circuit 255 can also be controlled via user interface 270.

Defibrillator 200 can optionally include a communication module 290, for establishing one or more wired or wireless communication links with other devices of other entities, such as a remote assistance center, Emergency Medical Services (EMS), and so on. Module 290 may also include a socket or a plug for wired communication, an antenna for wireless communication, a processor, and other sub-components as may be deemed necessary by a person skilled in the art. This way, data and commands can be communicated, such as patient data, event information, therapy attempted, CPR performance, system data, environmental data, and so on.

Defibrillator 200 can optionally include other components.

Returning to FIG. 1, in embodiments, one or more of the components of the shown WCD system have been customized for patient 82. This customization may include a number of aspects. For instance, support structure 170 can be fitted to the body of patient 82. For another instance, baseline physiological parameters of patient 82 can be measured, such as the heart rate of patient 82 while resting, while walking, motion detector outputs while walking, etc. Such baseline physiological parameters can be used to customize the WCD system, in order to make its diagnoses more accurate, since bodies behave differently. For example, such parameters can be stored in a memory of the WCD system, and so on.

A programming interface can be made according to embodiments, which receives such measured baseline physiological parameters. Such a programming interface may input automatically in the WCD system the baseline physiological parameters, along with other data.

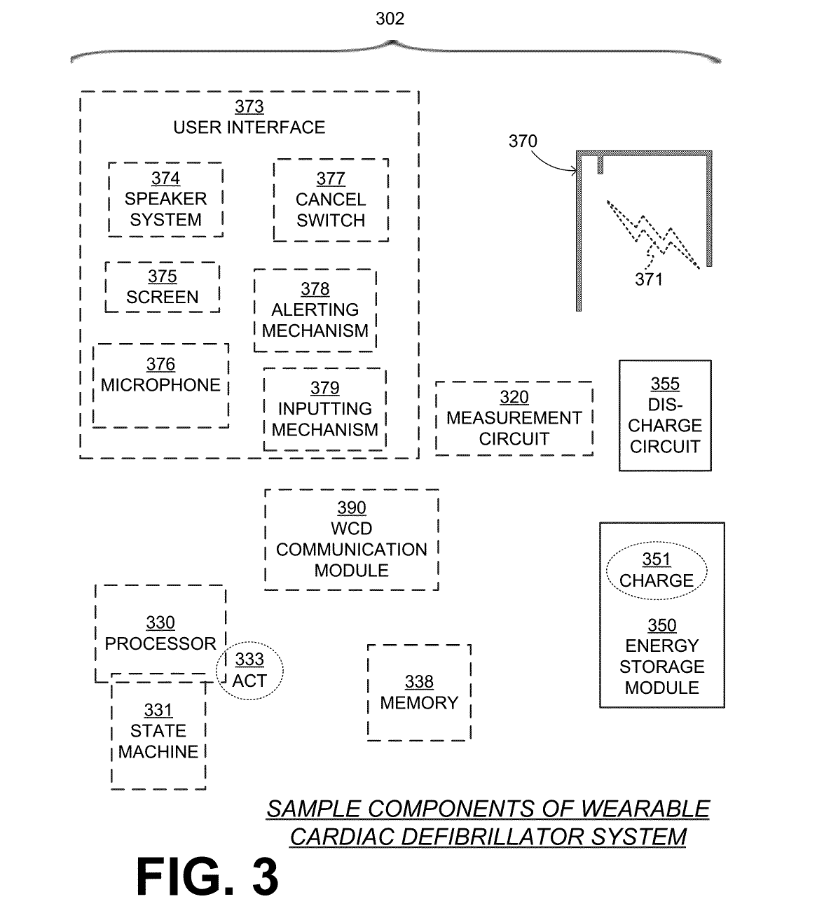

FIG. 3 is a diagram showing a collection 302 of sample components of Wearable Cardiac Defibrillator (WCD) systems, of which some are included in embodiments described in this document. The components of collection 302 may be included as described here, and sometimes with further modification for the individual embodiments, as will be understood from the present description.

Collection 302 includes a support structure 370 that is configured to be worn by a patient, who is not shown. Support structure 370 is shown only generically in FIG. 3, and can be made as described for support structure 170. In addition, collection 302 includes an energy storage module 350, which can be configured to be coupled to support structure 370. Energy storage module 350 can be made as described for energy storage module 250, and can be configured to store an electrical charge 351. Moreover, collection 302 includes a discharge circuit 355, which can be configured to be coupled to energy storage module 350. Discharge circuit 355 can be made as described for discharge circuit 255, and can be configured to discharge electrical charge 351 through the patient. Accordingly, discharge 371 may take place while support structure 370 is worn by the patient.

Collection 302 also includes a measurement circuit 320, a processor 330, and a memory 338, which can be made as described respectively for measurement circuit 220, processor 230, and memory 238. Measurement circuit 320 can be configured to render a physiological input from a patient physiological signal, such as an ECG. It can also be configured to render the patient impedance.

Processor 330 can be coupled to support structure 370, for example by being implemented within one of the larger components of a WCD system as shown. Processor 330 may control various components of a WCD system according to embodiments, even if not referred to explicitly. Moreover, processor 330 can be configured to determine from the physiological input of measurement circuit 320 whether or not a shock condition is met. If it is met, discharge circuit 355 can be configured to discharge electrical charge 351 through the patient. In addition, a state machine 331 may indicate an internal state of a WCD system, and may be implemented within processor 330, in conjunction with it, or otherwise, as is known.

Processor 330 can be configured to perform one or more internal acts 333 for its various functions. These may change the state of a state machine, such a state machine 331. Or, an internal act may be performed because of a changed state in the state machine.

One of these internal acts 333 can be for ultimately causing a speaker, such as speaker of speaker system 374, to output a sound. The sound would be a message to the wearer appropriate to the circumstances. The message could be in emergency situations, or to confirm to the user that all is operational or if an issue is detected. The acts can invoke appropriate parts of a memory, such as memory 338, where prompts can be stored. Relevant prompts that can be stored can include such as CPR related commands and tones, metronome, depth or other CPR-related feedback, ventilation guidance, etc. Moreover, these prompts or alerts may be stored in different languages, and the language may be selectable from an interface, along with other patient settings. The language may be selectable from the factory at the time of ordering, or at the time of fitting, or at the time of normally wearing by the patient, or at an emergency time by a bystander (for example between English and Spanish). Other items storable in the memory could be recordings of scene audio, and recordings of background sounds.

Collection 302 additionally includes a user interface 373. In embodiments, user interface 373 can be made as described for user interface 270. In embodiments, user interface 373 may be configured to be coupled to support structure 370, in which case components of user interface 373 can therefore be configured to be coupled to support structure 370. Coupling can be performed by the user, or by the manufacturer. If user interface 373 is coupled to support structure 370 by the manufacturer, these elements will be provided as coupled or even attached to each other. In embodiments, user interface 373 can be configured to input data files by having appropriate input devices, such as ports for data, etc.

In embodiments, user interface 373 may include a speaker system 374 that includes one or more speakers capable of outputting one or more sounds. The speakers may be located at appropriate places with respect to the body depending on the intended audience. For example, one or more speakers may be located on outer garment 99, such as at the lapel for audio intended for the patient, and on the chest facing outward intended for bystanders.

In embodiments, user interface 373 may also include a screen 375 that can be configured to display information. In some embodiments, screen 375 is a touch screen that may also receive usage inputs.

In embodiments, user interface 373 may further include a microphone 376 that can be configured to sense one or more ambient sounds. Microphone 376 can be used in any number of ways, for example recording scene audio, recording responses by the patient, etc. Noise cancellation technology may also be provided for microphone 376, especially if the latter is provided close to a speaker.

Microphone 376 can be configured to be recording always, or to start recording upon a specific event. The specific event may be, for example, actuating a switch or by saying one or more specific words that are recognizable, such as “HELP ME”. Thus, microphone 376 can be configured to sense ambient sounds that may include one or more preset words. In addition, a memory such as memory 338 can be configured to record the sensed ambient sound occurring after the one or more preset words, but not before. Additional controls may stop and restart the recording.

User interface 373 sometimes includes a cancel switch 377 for operations as described above. More particularly, cancel switch 377 can be configured to be actuated; and if it is actuated, it can prevent electrical charge 351 from being discharged, even if the shock condition is met. Cancel switch 377 can be implemented as a mechanical pushbutton, a switch, a button displayed on screen 375, a keypad into which a special code may be input, a fingerprint reader, and so on. When discharge 371 is imminent, the patient may be warned and be given a limited time to respond, in order to prove that discharge 371 is not necessary. This response can be made by actuating cancel switch 377. In some embodiments, cancel switch 377 and other switches intended for quick reaction are integrated into the speaker, or close to the speaker, or close to a vibration device. When the patient hears a sound from a speaker or feels the vibration, the reflex of reaching for the sound or the vibration would also be the right one for entering a response for the WCD system.

User interface 373 sometimes includes an alerting mechanism 378. Alerting mechanism 378 can be configured to attract the attention of the patient when activated. Such can be useful when needing the patient to respond, for example for helping with a diagnosis. In some embodiments, alerting mechanism 378 is tactile, and can vibrate. In other embodiments, alerting mechanism 378 can be part of speaker system 374, and so on.

User interface 373 sometimes includes an inputting mechanism 379. This can be implemented by any of the mechanisms described for user interface 270, which permit a patient or a bystander to enter inputs that are also sometimes called usage inputs. Sample usage inputs can be in an emergency, or to trigger status update/check. Sample inputting mechanisms can be buttons, knobs, switches, touch screens, etc. In some embodiments, cancel switch 377 is an example of inputting mechanism 379. In addition, for a patient with manual dexterity limitations, voice input via microphone 376 may be preferable over operating inputting mechanism 379 manually.

Collection 302 further includes a WCD communication module 390, which can be made for wired or wireless communication, for example as described for communication module 290. WCD communication module 390 can be configured to be coupled to support structure 370. WCD communication module 390 can be controlled by processor 330. For example, when processor 330 performs a specific internal act for causing a speaker to output a sound, WCD communication module 390 can be configured to generate a prompt signal responsive to the internal act and, ultimately, the speaker may output the sound responsive to the prompt signal. The prompt signal can be carried by wired or wireless communication. For wireless communication, WCD communication module 390 may be able to establish one or more wireless communication links with other compatible devices.

The above-mentioned components may be implemented in suitable places of a WCD system according to embodiments. Some of them can be implemented within monitoring device 180 or monitoring device 281.

In some embodiments, a speaker is implemented in a head component. Examples are now described.

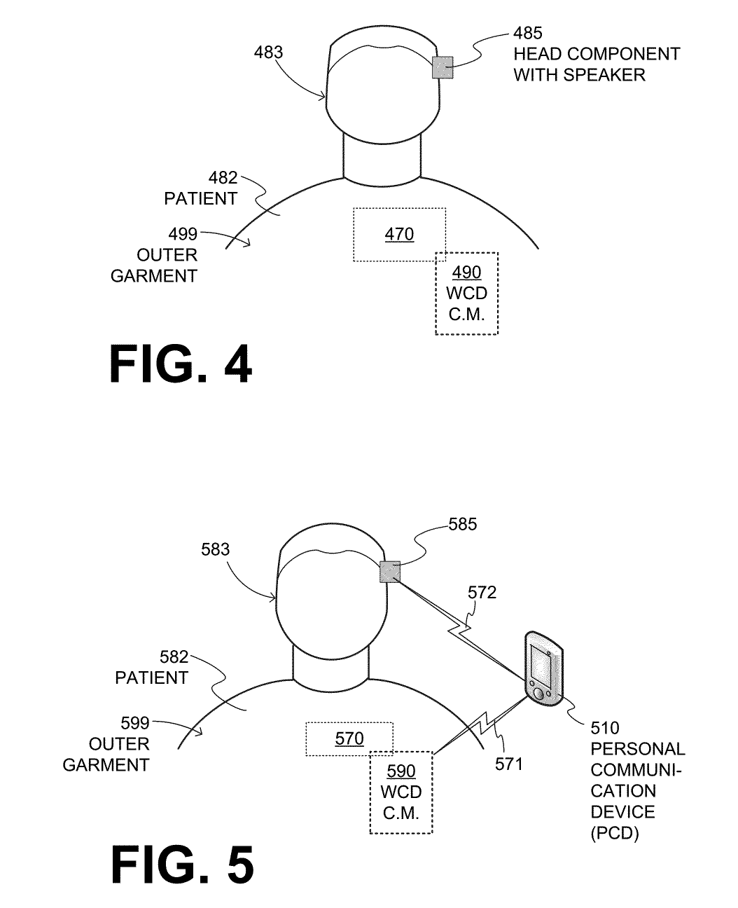

FIG. 4 is a diagram of a patient 482, who is wearing an outer garment 499. Patient 482 is wearing components of a WCD system made according to embodiments. Only a few of these components are shown for brevity. These components may include a support structure 470 that can be as described for support structure 370. Support structure 470 is shown in dotted lines because it is under outer garment 499 in this example.

The components of the WCD system of FIG. 4 may also include a head component 485, which is configured to be worn at the head 483 of patient 482. Head component 485 can include a speaker that is not shown separately, and can be made as one of the speakers of speaker system 374. Having the speaker of head component 485 near the ear of patient 482 will permit the sound to be at a lower intensity, for more discreet operation. Another term for intensity is sound pressure level.

Head component 485 may be implemented in any number of ways. In some embodiments, it is a headset, such as a Bluetooth headset. In some embodiments, head component 485 is supported at least in part on the nose of patient 482, for example by being a set of glasses. In some embodiments, head component 485 is configured to be placed in or on the ear of patient 482, for example by being an earpiece or something akin to a hearing aid.

The components of the WCD system of FIG. 4 may further include an energy storage module (not shown), a discharge circuit (not shown), and a processor (not shown), which can be as described respectively for energy storage module 350, discharge circuit 355, and processor 330. The processor can be configured to perform an internal act for ultimately causing the speaker of head component 485 to output a sound. Conversely, the speaker of head component 485 can be configured to output a sound responsive to the internal act performed by the processor.

The sound can be caused to be output in a number of ways. For instance, the components of the WCD system of FIG. 4 may additionally include a WCD communication module 490, which can be made as WCD communication module 390. WCD communication module 490 can be for wired or wireless communication. WCD communication module 490 may be configured to generate a prompt signal responsive to the internal act by the processor.

In wired embodiments, the components of the WCD system of FIG. 4 may furthermore include at least one electrical wire (not shown) physically coupled between WCD communication module 490 and head component 485, so as to carry the prompt signal. In these embodiments, the sound can be output by the speaker responsive to the prompt signal that is carried by the wire and received via the wire. In such embodiments, WCD communication module 490 may optionally include a socket, and the electrical wire may terminate in a plug that can be plugged into the socket.

In wireless embodiments, WCD communication module 490 can be further configured to transmit wirelessly the prompt signal it generates, for example using Bluetooth. In these embodiments, head component 485 is configured to receive the transmitted prompt signal, for example by having an antenna, etc. Again, the sound can be output responsive to the received prompt signal.

In some embodiments, an additional Personal Communication Device (PCD) is used. An example is now described.

FIG. 5 is a diagram of a patient 582, who is wearing an outer garment 599. Patient 582 is wearing components of a WCD system made according to embodiments. Only a few of these components are shown for brevity. These components may include a support structure 570 that can be as described for support structure 370. Support structure 570 is shown in dotted lines because it is under outer garment 599.

The components of the WCD system of FIG. 5 may also include a head component 585, which is configured to be worn at the head 583 of patient 582. Head component 585 can include a speaker that is not shown separately, and can be made as one of the speakers of speaker system 374. Head component 585 may be implemented in any number of ways, for example as described for head component 485.

The components of the WCD system of FIG. 5 may further include an energy storage module (not shown), a discharge circuit (not shown), and a processor (not shown), which can be as described respectively for energy storage module 350, discharge circuit 355, and processor 330. The processor can be configured to perform an internal act for ultimately causing the speaker of head component 585 to output a sound. Conversely, the speaker of head component 585 can be configured to output a sound responsive to the internal act performed by the processor.

The sound can be caused to be output in ways that include a Personal Communication Device (PCD) 510, which is described in more detail later in this document. In particular, the components of the WCD system of FIG. 5 may additionally include a WCD communication module 590, which can be made as WCD communication module 390. WCD communication module 590 may be configured to generate a prompt signal responsive to the internal act by the processor, and to further transmit it wirelessly.

In these embodiments, PCD 510 is configured to receive the transmitted prompt signal from WCD communication module 590. In addition, PCD 510 is configured to transmit wirelessly an action signal responsive to receiving the transmitted prompt signal. Head component 585 is configured to receive the transmitted action signal, for example by having an antenna, etc. Again, the sound can be output responsive to the received action signal. These wireless transmissions are preferably done after pairing, e.g. establishing communication links such as links 571, 572.

PCD 510 is now described in more detail. In some embodiments, the PCD is a custom device that is part of the WCD system. In other embodiments, the PCD is an otherwise commercially available mobile communication device that is intended to be used together with a WCD system. Examples include a smartphone, a tablet, a device that can be strapped to a person's wrist such as a smartwatch, etc. Using them together can be accomplished, for example, by downloading to the mobile communication device a suitable application (“app”), pairing it with the WCD system, etc. When operating, PCD 510 could have different interfaces for provide audio such to a headset, to a speaker, to earphones, etc.

In additional embodiments, the audio signals may also be sent to other paired output devices beyond the patient. For example, in a room where audio system is present, some alerts may be “broadcasted” to the audio system or to a TV display, or to the sound system of an automobile. Or, the PCD could be a two-way communication system.

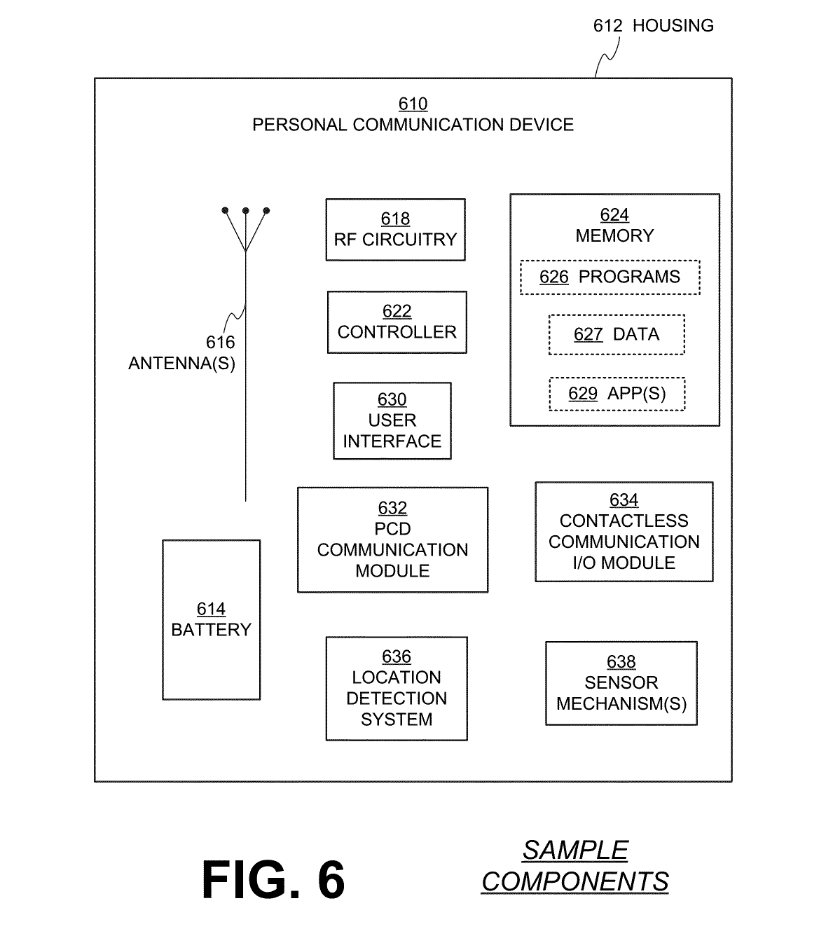

FIG. 6 is a diagram showing possible sample components of a personal communication device (PCD) 610. PCD 610 could be the same as PCD 510 that is depicted in FIG. 5. PCD 610 can be provided with a housing 612, and its components are typically within housing 612. PCD 610 can be powered by a battery 614 that is rechargeable, for portability of mobile communication device 610.

PCD 610 may include one or more antennas 616 for wireless communication. PCD 610 may also include RF (radio frequency) circuitry 618. RF circuitry 618 cooperates with antenna(s) 616 to receive and send RF signals. RF circuitry 618 converts wired electrical signals to/from RF signals. RF circuitry 618 may include well-known circuitry for performing these functions, including but not limited to an RF transceiver, one or more amplifiers, a tuner, one or more oscillators, a digital signal processor, a CODEC chipset, a subscriber identity module (SIM) card, a memory, and so forth. The one or more antennas 616, plus RF circuitry 618, can establish comlinks as per the above, when cooperating with one of the communication modules of mobile communication device 610 that are described below.

PCD 610 can additionally include a controller 622, for controlling its operation. Controller 622 can be one or more processors, implemented as a Central Processing Unit (CPU), a digital signal processor, a microprocessor, a microcontroller, an application-specific integrated circuit (ASIC), a programmable logic device (PLD), or other implementation. Controller 622 can be optionally combined in a single chip with a memory controller and a peripherals interface.

PCD 610 may also include a memory 624, which can include both persistent/nonvolatile and non-persistent/volatile memory components. Memory 624 can be implemented in any technology for memory for such devices, for example RAM, ROM, EEPROM, flash memory, and so on. Additional memory components may be plugged in from a device external to housing 612, such as a thumb drive, in some embodiments. As such, memory 624 can include a non-transitory computer-readable storage medium. Memory 624 can store programs 626 and data 627. Programs 626 can include instructions that can be executed by controller 622. Programs 626 may include an operating system, such as, for example, Android, iOS, Windows Phone, Symbian, or BlackBerry OS.

In addition, one or more app(s) 629 can be stored in memory 624, as mentioned above. App(s) 629 can be common or special, and include instructions that can be executed by controller 622. Common app(s) 629 can be provided for a contacts module, an email client module, a calendar module, a camera module, a maps module, and so on. These can be adapted for use with a WCD system.

It will be appreciated that many of the methods of the invention can be performed by PCD 610 due to one or more special app(s) 629, in addition to common apps. Even if PCD 610 is initially provided in a more generic form without special app(s) 629, the latter may be downloaded later.

PCD 610 may further include a user interface 630, for use by a user, such as user 282. User interface 630 includes individual devices for receiving inputs by the user, generating outputs for the user, or both. The outputs for the user can be human-perceptible indications, such as sounds, vibrations, lights, images, and so on. Examples of such individual devices include a screen that could be a touch screen, a physical keypad, an optical finger interface, one or more speakers, one or more microphones, one or more accelerometers, and so on. Such devices can be included within housing 612, or can be added by a separate plugin, such as a keypad, a keyboard, a display, and a speaker.

PCD 610 can moreover include a PCD communication module 632. PCD communication module 632 can conduct communication using any one of a variety of standards, protocols and technologies. Examples of the latter are Global System for Mobile Communications (GSM), Enhanced Data GSM Environment (EDGE), high-speed downlink packet access (HSDPA), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS), and/or Short Message Service (SMS), or any other suitable communication protocol, including communication protocols not yet developed as of the filing date of this document.

Additionally, PCD 610 can include a contactless communication I/O module 634. Module 634 can be used for short range detection and communication, such as Near-Field Communications (NFC), Radio Frequency Identification (RFID), and so on.

PCD 610 can also include a location detection system 636, which can be implemented with GPS. PCD 610 can further include one or more sensor mechanisms 638. Mechanisms 638 can include one or more accelerometers, a proximity sensor, a magnetometer, optical sensors, an image sensor, and so on. Data captured by location detection system 636 and sensor mechanisms 638 can be stored as data 627.

Programs 626, and also app(s) 629, can advantageously coordinate their use with components of PCD 610. For example, and depending on the application, antenna(s) 616 may be used to detect electromagnetic fields (EMF), and a microphone of user interface 630 may be used to detect sound. Many of the components of a mobile communication device are well known in the field, and therefore and are not described further.

It will be appreciated that modes of PCD 610 can be used advantageously according to embodiments. For example, PCD 610 could be placed in a video mode for transmitting video in addition to transmitting voice as a telephone. Moreover, PCD 610 could be placed in an answer mode, so someone from another entity could talk with and listen to a bystander, without requiring the patient to answer the call.

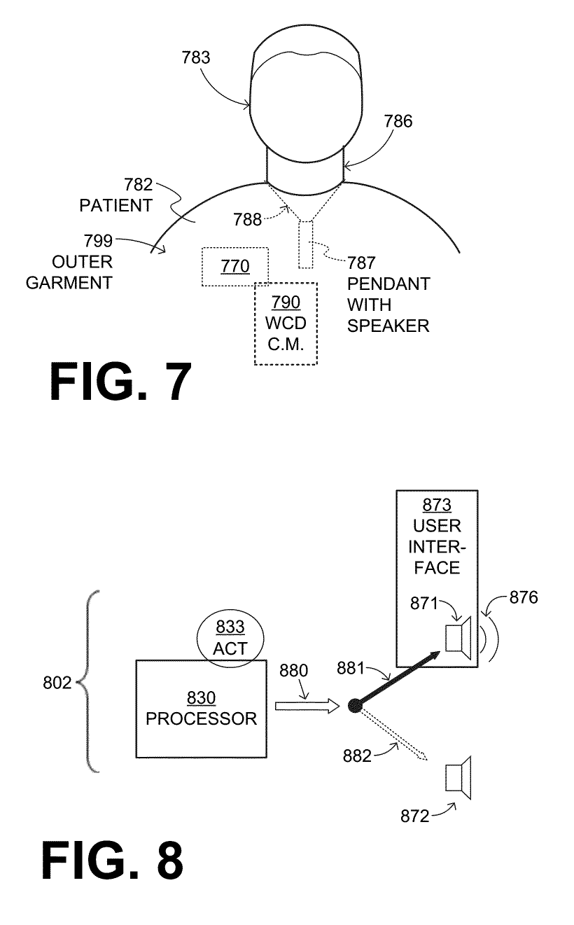

FIG. 7 is a diagram of a patient 782, who is wearing an outer garment 799. Patient 782 is wearing components of a WCD system made according to embodiments. Only a few of these components are shown for brevity. These components may include a support structure 770 that can be as described for support structure 370. Support structure 770 is shown in dotted lines because it is under outer garment 799 in this example.

The components of the WCD system of FIG. 7 may also include a pendant 787, which is physically uncoupled from support structure 770. Pendant 787 can be configured to be suspended from the neck 786 of patient 782, for example via a string 788. Pendant 787 can include a speaker that is not shown separately, and can be made as one of the speakers of speaker system 374.

The components of the WCD system of FIG. 7 may further include an energy storage module (not shown), a discharge circuit (not shown), and a processor (not shown), which can be as described respectively for energy storage module 350, discharge circuit 355, and processor 330. The processor can be configured to perform an internal act for ultimately causing the speaker of pendant 787 to output a sound. Conversely, the speaker of pendant 787 can be configured to output a sound responsive to the internal act performed by the processor.

The sound can be caused to be output in a number of ways. For instance, the components of the WCD system of FIG. 7 may additionally include a WCD communication module 790, which can be made as WCD communication module 390. WCD communication module 790 may be configured to generate a prompt signal responsive to the internal act by the processor, and to transmit it wirelessly. In these embodiments, pendant 787 is configured to receive the transmitted prompt signal, for example by having an antenna, etc. Again, the sound can be output responsive to the received prompt signal.

Pendant 787 may have additional functionalities. For example, it may include a vibration device for notifying the patient, a screen or a visual indicator for communicating information, and a switch for receiving a usage input.

FIG. 8 is a diagram of a set 802 of selected components of a sample wearable cardiac defibrillator (WCD) system according to embodiments. Set 802 includes a processor 830, which can be made as processor 330. The WCD system of FIG. 8 may further include other components that are not shown, such as a support structure, an energy storage module and a discharge circuit, which can be made as shown in collection 302 for respective elements. In addition, such a system may also include a microphone and its related functionality as described above.

In the WCD system of FIG. 8, processor 830 can be coupled to the support structure. Processor 830 may be configured to perform an internal act 833, which can be as described for internal act 333. This WCD system also includes a user interface 873, which can be as was described for user interface 373. User interface 873 can include a first speaker 871. First speaker 871 can be configured for use with a second speaker 872.

In some embodiments, second speaker 872 is not part of the WCD system. For example, second speaker 872 could be part of a commercially available device that is used as a personal communication device by the WCD system.

In other embodiments, second speaker 872 is part of the WCD system. For example, second speaker 872 can be part of user interface 873. Or, second speaker 872 is part of a custom personal communication device that is part of the WCD system.

In such embodiments, speakers 871, 872 may be tasked differently depending on the message or notification that is to be output. Accordingly, internal act 833 may belong in one of a first category or a second category of possible internal acts, or possibly in an additional category. For example, first speaker 871 can be configured to output a sound 876, responsive to internal act 833, and if internal act 833 belongs in the first category. And second speaker 872 can be configured to output a sound (not shown) responsive to internal act 833, and if internal act 833 belongs in the second category. In the example of FIG. 8, first speaker 871 outputs a sound 876, according to a path 880, 881 shown from processor 830 to first speaker 871. The alternate path would have been 880, 882, and a “fork” in the road is shown as a switch, where the path continues from 880 into 881 towards first speaker 871, or into 882 towards second speaker 872. Of course, it will be understood that these conceptually shown paths can be implemented by methods or processes branching, and/or signals following different paths, whether wired or wireless, etc.

The classification into categories may be static or dynamic. For example, in some embodiments, a certain one of these internal acts, such as act 833, always belongs in the same one of these categories. In some embodiments, a certain one of these internal acts, such as act 833, may belong in the first or in the second category, depending on whether a condition flag is set. The condition flag may be based on the criticality of the event, or the intended recipient (bystander vs patient) of a prompt. Accordingly, different communication modes and even speakers may be employed for different users and situations, based on alert levels and criticality. Customization and configurability may permit better communication awareness and effectiveness.

In some embodiments, a WCD system such as that of FIG. 4, 5, 7 or 8 further includes a microphone (not shown in FIG. 4, 5, 7 or 8), which can be made as microphone 376. The microphone can be located anywhere in the WCD system. In some embodiments, head component 485 or 585 further includes the microphone.

In some embodiments, the microphone detects a level of an ambient sound, for example from a metric such as average intensity in certain frequencies, and so on. In such embodiments, the sound output by the speaker may have an intensity that is adjusted according to the detected level of the ambient sound. For example, the output sound may be louder if the ambient sound is louder, the speaker may become covered by outer garment 99 and thus muffled, and so on.

In addition, ambient sound may be analyzed for its frequency content, and the output sound can be in a set of adjusted frequencies so as to avoid the ambient sound. The output sound may be available in sets that have different main frequency contents, and the right set can be selected based on the detected ambient sound. This way the sound might not need to be much louder, even though it may be different.

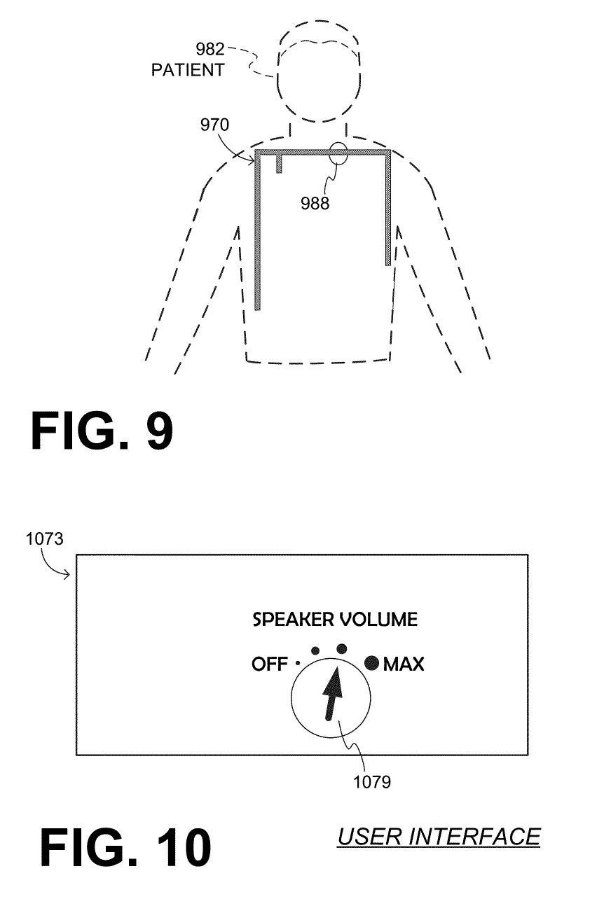

FIG. 9 is a diagram of a patient 982, who is not wearing an outer garment. Patient 982 is wearing components of a WCD system made according to embodiments. Only a few of these components are shown for brevity. These components may include a generic support structure 970 that can be as described for support structure 370. Support structure 970 is further configured to be worn by the patient under tension. This means that at least a portion of support structure 970 presses against at least a portion of the body of the patient. This pressure, and resulting tension, can be implemented in any number of ways, such as with a harness or belt or garment having an elastic portion that is stretched when it is worn, and in a number of different locations of the body.

The components of the WCD system of FIG. 9 may also include a speaker 988, which can be made as one of the speakers of speaker system 374. Speaker 988 can be configured to output a sound.

Speaker 988 can be coupled to support structure 970 such that the tension physically biases the speaker towards a body of the patient when the support structure is worn by a patient. In the example of FIG. 9, this is illustrated conceptually by showing speaker 988 be under a horizontal portion of generic support structure 970, being pressed at a generic location. Other locations are also possible.

Accordingly, the sound output by speaker 988 is transmitted into the body of patient 982. This can be implemented in a number of ways. For example, speaker 988 can be located with respect to support structure 970 such that it is physically biased towards a bone or the clavicle of patient 982. Biasing need not be with much force—it may have the feeling of wearing a tight sweater. More particularly, the intent is to have the speaker firmly there, not losing contact with the shifting of the patient's body. The transmission of sound waves via bone conduction may improve the patient's awareness, reception of the message, and response.

The transmitted sound can be buzzing, and so on. In some embodiments, the sound includes a voice that speaks one or more words.

The components of the WCD system of FIG. 9 may further include an energy storage module (not shown), and a discharge circuit (not shown), which can be as described respectively for energy storage module 350 and discharge circuit 355.

In some embodiments, the components of the WCD system of FIG. 9 may further include a processor (not shown), which can be as described respectively for processor 330. In such embodiments, the speaker can be configured to output the sound responsive to the internal act performed by the processor.

Examples of volume adjustment are now described according to embodiments. It will be understood that these examples apply to a WCD system having components from collection 302, as well as to the more particular embodiments described in connection with FIGS. 4, 5, 7 and 9.

FIG. 10 is a diagram of a portion of sample user interface 1073 made according to an embodiment of a wearable cardiac defibrillator (WCD) system. In some respects, user interface 1073 can be made as user interface 373. In addition, user interface 1073 is configured to receive a volume adjustment input from a knob or dial 1079. The volume adjustment input can be received from the patient while the support structure is worn by the patient, and could be one of the earlier-mentioned usage inputs. In this example, the volume adjustment input can have different values, such as a first value, a second value, etc., which are detected when dial 1079 is rotated from respectively a first position to a second, etc. Such a volume adjustment input may be intended to adjust the volume of the sound from the speaker.

The volume adjustment input can be received in any number of ways, and the way of FIG. 10 was only an example. For instance, the user interface could include a screen, a button, a knob or a dial as shown, a switch, etc. In some embodiments the user interface is on the WCD system, and the volume adjustment input is received from direct action on the WCD system. In some embodiments the volume adjustment input is received wirelessly from a personal communication device (PCD), such as was described above.

Examples of muting are now described according to embodiments. It will be understood that these examples apply to a WCD system having components from collection 302, as well as to the more particular embodiments described in connection with FIGS. 4, 5, 7 and 9.

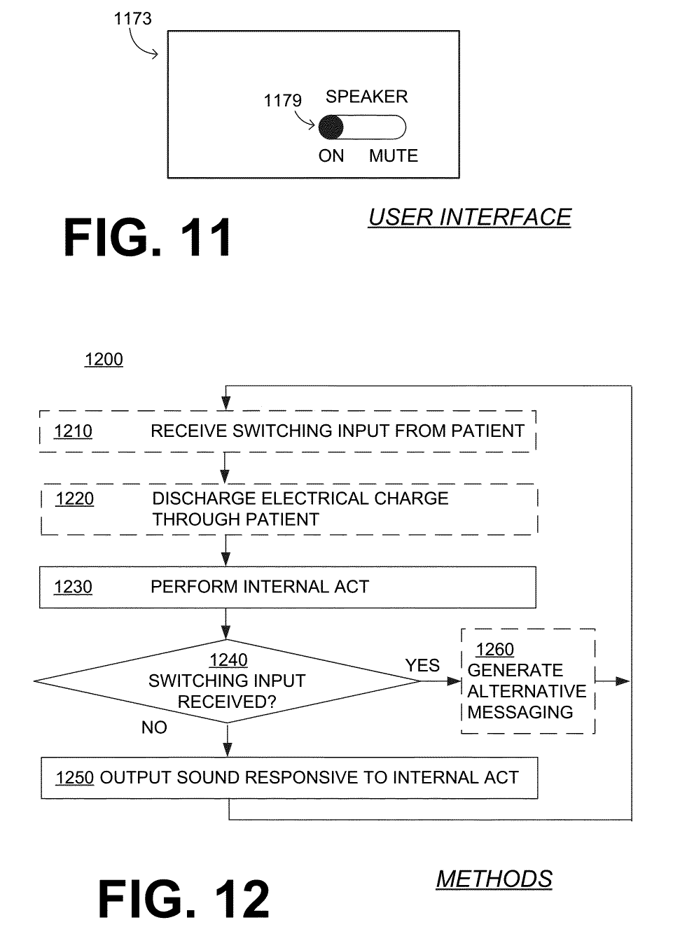

FIG. 11 is a diagram of a portion of sample user interface 1173 made according to an embodiment of a wearable cardiac defibrillator (WCD) system. In some respects, user interface 1173 can be made as user interface 373. In addition, user interface 1173 is configured to receive a switching input from a switch 1179. The switching input can be received from the patient while the support structure is worn by the patient, and could be one of the earlier-mentioned usage inputs. In this example, the switching input can be when switch 1179 for the “SPEAKER” is moved from its shown “ON” position to the “MUTE” position. Such a switching input can be received by detecting the position of switch 1179, and may be intended to mute the sound from the speaker. In such embodiments, if the switching input has been received, the sound can be caused to not be output responsive to the internal act being performed by the processor. Of course, the switching input is reversible, etc.

The switching input can be received in any number of ways, and the way of FIG. 11 was only an example. For instance, the user interface could include a screen, and a status indicator on the screen of whether the muting input has been received. In some embodiments, the user interface could include a button, a knob, a switch as shown, or an on-screen input. In some embodiments the user interface is on the WCD system, and the switching input is received from direct action on the WCD system. In some embodiments the switching input is received wirelessly from a personal communication device (PCD), such as was described above.

In some embodiments, only some of the possible prompts may become muted, while others might not be mutable. For example it is possible to not have a critical alarm be able to be silenced because, after all, it is preferable to warn bystanders to stand back before the patient is shocked. Less critical communications, however, such as status updates can be silenced. Accordingly, the internal act that the processor can perform for generating a notification by the speaker may belong in a first category or in a second category of possible corresponding internal acts. Only acts in the first category need be mutable. As such, if the internal act is performed and the switching input has been received, the sound can be caused to not be output responsive to the internal act if the internal act belongs in the first category, but is output if the internal act belongs in the second category. Also, some categories of prompts may be at different volume levels than others.

In some embodiments there are one or more auxiliary devices for notifying the patient, in addition to the notification by the speaker outputting the sound. Such an auxiliary device may be operative at various times or always, perhaps depending on the importance and urgency of the communication.

There can be a number of such auxiliary devices. For example, a WCD system may further have a vibration device coupled to the support structure, or the user interface may include a visual device. Such can be respectively configured to vibrate or generate a visual input responsive to the internal act that is performed by the processor, which generates the notification.

In some embodiments, such an auxiliary device becomes operative only when the sound from the speaker is muted. To continue the examples immediately above, the vibration device may be configured to vibrate, or the visual device may be configured generate a visual input, responsive to the internal act only if the switching input has been received.

The devices and/or systems mentioned in this document perform functions, processes and/or methods. These functions, processes and/or methods may be implemented by one or more devices that include logic circuitry. Such a device can be alternately called a computer, and so on. It may be a standalone device or computer, such as a general purpose computer, or part of a device that has one or more additional functions. The logic circuitry may include a processor and non-transitory computer-readable storage media, such as memories, of the type described elsewhere in this document. Often, for the sake of convenience only, it is preferred to implement and describe a program as various interconnected distinct software modules or features. These, along with data are individually and also collectively known as software. In some instances, software is combined with hardware, in a mix called firmware.

Moreover, methods and algorithms are described below. These methods and algorithms are not necessarily inherently associated with any particular logic device or other apparatus. Rather, they are advantageously implemented by programs for use by a computing machine, such as a general-purpose computer, a special purpose computer, a microprocessor, a processor such as described elsewhere in this document, and so on.

This detailed description includes flowcharts, display images, algorithms, and symbolic representations of program operations within at least one computer readable medium. An economy is achieved in that a single set of flowcharts is used to describe both programs, and also methods. So, while flowcharts described methods in terms of boxes, they also concurrently describe programs.

In embodiments, FIG. 12 shows a flowchart 1200 for describing methods according to embodiments. The methods of flowchart 1200 may also be practiced by embodiments described elsewhere in this document.

According to an optional operation 1210, a switching input may be received from a patient wearing a WCD system. The switching input may be received via the user interface of the WCD system, while a support structure of the WCD and possibly other components are worn by the patient.

According to another, optional operation 1220, an electrical charge stored in an energy storage module of the WCD system may be discharged through the patient, while the support structure is worn by the patient. Discharging may take place via a discharge circuit of the WCD system.

According to another operation 1230, an internal act is performed. The internal act can be performed by a processor of the WCD system, such as processor 330 of collection 302. The internal act can be of the type intended to generate a verbal communication to the patient, such as a notification by setting a flag, setting a parameter, and so on. The internal act can be in response to a change in a state machine, such as state machine 331.

According to another operation 1240, it can be inquired whether a switching input has been received. The switching input may have been received as seen above, for example, in operation 1210.

If, at operation 1240, the answer is no, then according to another operation 1250, a sound can be output via a speaker of the WCD system, while the support structure is worn by the patient. The sound can be output responsive to the internal act performed by the processor, and due to the fact that the switching input that would mute the speaker has not been received. Execution may then return to operation 1210.

If, at operation 1240, the answer is yes, it means that the speaker is intended to not be heard by the patient. Then, according to another, optional operation 1260, alternative messaging may be generated and execution may then return to operation 1210. In other words, the internal act will have been performed at operation 1230, but the sound would not be output responsive to the internal act since operation 1250 is bypassed.

The alternative messaging of operation 1260 may be implemented by using an auxiliary device, such as was described above. For example, the WCD system could further include a vibration device that vibrates responsive to the internal act only if the switching input has been received. Or, the WCD system could further include a visual device that generates a visual input responsive to the internal act only if the switching input has been received. As another variation, such an auxiliary device could operate regardless of whether the switching input has been received or not.

The methods of flowchart 1200 describe a possible mode, which can be executed in a number of different passes, and different sequences can be considered, where optional operations may or may not be executed. For example, execution could be in the following order of operations: 1230, 1240 (with the answer being no), 1250, 1210, 1230, 1240 (with the answer being yes, which means operation 1250 will not executed), 1220.

In the methods described above, each operation can be performed as an affirmative step of doing, or causing to happen, what is written that can take place. Such doing or causing to happen can be by the whole system or device, or just one or more components of it. In addition, the order of operations is not constrained to what is shown, and different orders may be possible according to different embodiments. Moreover, in certain embodiments, new operations may be added, or individual operations may be modified or deleted. The added operations can be, for example, from what is mentioned while primarily describing a different system, apparatus, device or method.

A person skilled in the art will be able to practice the present invention in view of this description, which is to be taken as a whole. Details have been included to provide a thorough understanding. In other instances, well-known aspects have not been described, in order to not obscure unnecessarily the present invention. Plus, any reference to any prior art in this description is not, and should not be taken as, an acknowledgement or any form of suggestion that this prior art forms parts of the common general knowledge in any country.

This description includes one or more examples, but that does not limit how the invention may be practiced. Indeed, examples or embodiments of the invention may be practiced according to what is described, or yet differently, and also in conjunction with other present or future technologies. Other embodiments include combinations and sub-combinations of features described herein, including for example, embodiments that are equivalent to: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In this document, the phrases “constructed to” and/or “configured to” denote one or more actual states of construction and/or configuration that is fundamentally tied to physical characteristics of the element or feature preceding these phrases and, as such, reach well beyond merely describing an intended use. Any such elements or features can be implemented in any number of ways, as will be apparent to a person skilled in the art after reviewing the present disclosure, beyond any examples shown in this document.

The following claims define certain combinations and subcombinations of elements, features and steps or operations, which are regarded as novel and non-obvious. Additional claims for other such combinations and subcombinations may be presented in this or a related document.

1. A wearable cardiac defibrillator (WCD) system, comprising:
a support structure configured to be worn by a patient;
an energy storage module configured to be coupled to the support structure and to store an electrical charge;
a discharge circuit configured to be coupled to the energy storage module and configured to discharge the electrical charge through the patient while the support structure is worn by the patient;
a processor coupled to the support structure and configured to perform an internal act; and
a head component configured to be worn at a head of the patient, the head component including a speaker configured to output a sound responsive to the internal act.

2. The WCD system of claim 1, in which
the head component is supported at least in part on a nose of the patient.

3. The WCD system of claim 1, in which
the head component is configured to be placed in or on an ear of the patient.

4. The WCD system of claim 1, further comprising:
a WCD communication module configured to generate a prompt signal responsive to the internal act; and
at least one electrical wire physically coupled between the WCD communication module and the head component so as to carry the prompt signal, and
in which the sound is output responsive to the carried prompt signal.

5. The WCD system of claim 4, in which
the WCD communication module includes a socket, and
the electrical wire terminates in a plug that can be plugged into the socket.

6. The WCD system of claim 1, further comprising:
a WCD communication module configured to generate a prompt signal responsive to the internal act and to transmit wirelessly the prompt signal, and
in which the head component is configured to receive the transmitted prompt signal, and
the sound is output responsive to the received prompt signal.

7. The WCD system of claim 1, intended for use with a personal communication device (PCD), the WCD system of claim 1 further comprising:
a WCD communication module configured to generate a prompt signal responsive to the internal act, and to transmit wirelessly the prompt signal, and
in which the PCD is configured to receive the transmitted prompt signal and transmit an action signal responsive to receiving the transmitted prompt signal,
the head component is configured to receive the transmitted action signal, and
the sound is output responsive to the received action signal.

8. The WCD system of claim 7, further comprising:
the PCD.

9. The WCD system of claim 7, further comprising:
the PCD, and
in which the PCD is strapped to the patient's wrist.

10. The WCD system of claim 1, further comprising:
a microphone.

11. The WCD system of claim 10, in which
the head component further includes the microphone.

12. The WCD system of claim 10, in which
the microphone is configured to start recording upon sensing the sound of one or more preset words.

13. The WCD system of claim 10, in which
the microphone detects a level of an ambient sound, and
the sound has an intensity adjusted according to the detected level of the ambient sound.

14. The WCD system of claim 1, further comprising:
a user interface configured to receive a volume adjustment input from the patient while the support structure is worn by the patient, and
in which, if the volume adjustment input has a first value, the sound is output at a first intensity, but if the volume adjustment input has a second value different from the first value, the sound is output at a second intensity different from the first intensity.

15. The WCD system of claim 14, in which
the volume adjustment input is received wirelessly from a personal communication device (PCD).

16. The WCD system of claim 1, further comprising:
a user interface configured to receive a switching input from the patient while the support structure is worn by the patient, and
in which, if the switching input has been received, the sound is caused to not be output responsive to the internal act.

17. The WCD system of claim 16, in which
the switching input is received wirelessly from a personal communication device (PCD).

18-73. (canceled)
